(19) United States
(12) Patent Application Publication
(10) Pub. No.: US 2024/0358870 A1
(43) Pub. Date: Oct. 31, 2024

(54) DISINFECTION STATION FOR A STORAGE CONTAINER

(71) Applicant: Autostore Technology AS, Nedre Vats (NO)

(72) Inventor: Kjetil Gilje, Sandeid (NO)

(73) Assignee: Autostore Technology AS, Nedre Vats (NO)

(21) Appl. No.: 18/685,697

(22) PCT Filed: Aug. 22, 2022
(86) PCT No.: PCT/EP2022/073291

§ 371 Date: Feb. 22, 2024

(30) Foreign Application Priority Data
Aug. 23, 2021 (NO) 20211010

(51) Int. Cl. A61L 2/18; A61L 2/24; A61L 2/26; B65G 1/04

(52) U.S. Cl. CPC A61L 2/18; A61L 2/24; A61L 2/26; B65G 1/0464; A61L 2202/14; A61L 2202/23; B65G 2207/26

(57) ABSTRACT

A disinfection station for disinfection of a storage container in an automated storage and retrieval system includes a frame and a disinfection system. The frame includes a support, by which the storage container is supported in a disinfection position during disinfection, and an opening through which the storage container is transported between a storage position in the automated storage and retrieval system and the disinfection position. The disinfection system is for disinfecting the storage container in the disinfection position. The disinfection system includes a disinfection fluid applicator, a disinfection fluid container, and a pump controlled to pump the disinfection fluid from the container to the applicator. The opening is a top opening. The storage container is lowered through the top opening to the support before disinfection. The disinfection station includes a splash cover for protecting the surroundings from disinfection fluid. The disinfection station includes a collector for collecting excessive disinfection fluid.

### FIELD OF THE INVENTION

The present invention relates to a disinfection station for disinfection of a storage container in an automated storage and retrieval system. The present invention also relates to an access station for presentation of a storage container retrieved from an automated storage and retrieval system to a picker. The present invention also relates to an automated storage and retrieval system comprising a framework structure. The present invention also relates to a method for disinfection of a storage container in an automated storage and retrieval system.

### BACKGROUND AND PRIOR ART

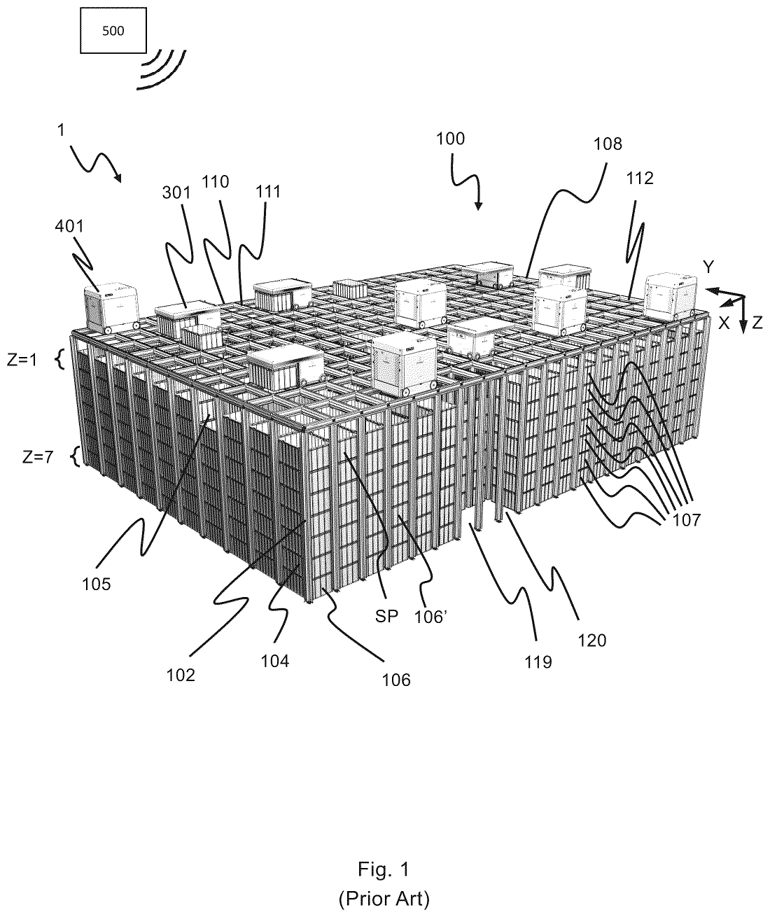

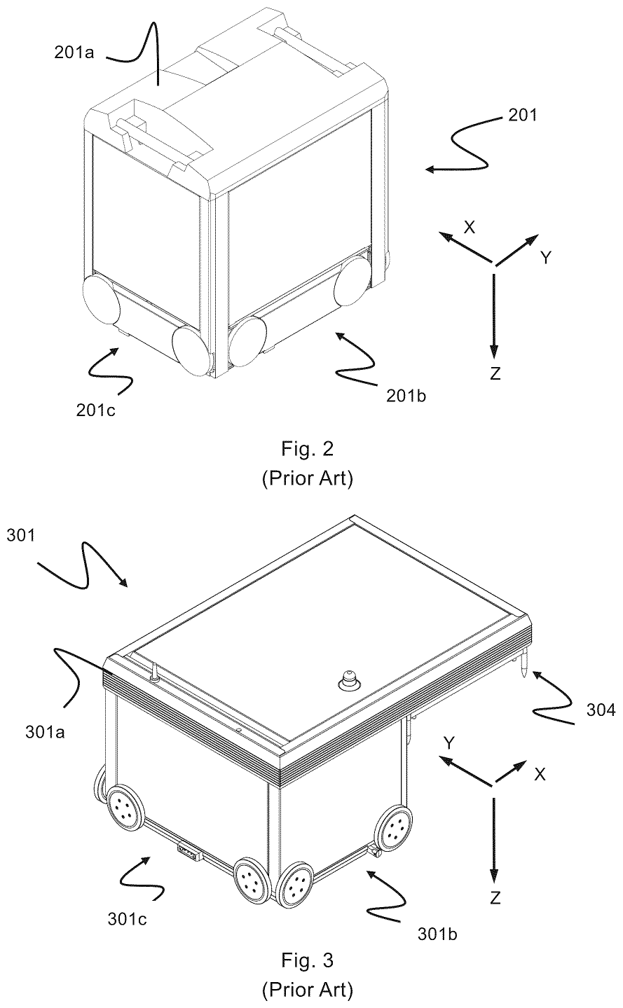

FIG. 1 discloses a prior art automated storage and retrieval system 1 with a framework structure 100 and FIGS. 2, 3 and 4 disclose three different prior art container handling vehicles 201,301,401 suitable for operating on such a system 1.

The framework structure 100 comprises upright members 102 and a storage volume comprising storage columns 105 arranged in rows between the upright members 102. In these storage columns 105 storage containers 106, also known as bins, are stacked one on top of one another to form stacks 107. The members 102 may typically be made of metal, e.g. extruded aluminum profiles.

The framework structure 100 of the automated storage and retrieval system 1 comprises a rail system 108 arranged across the top of framework structure 100, on which rail system 108 a plurality of container handling vehicles 201,301,401 may be operated to raise storage containers 106 from, and lower storage containers 106 into, the storage columns 105, and also to transport the storage containers 106 above the storage columns 105. The rail system 108 comprises a first set of parallel rails 110 arranged to guide movement of the container handling vehicles 201,301,401 in a first direction X across the top of the frame structure 100, and a second set of parallel rails 111 arranged perpendicular to the first set of rails 110 to guide movement of the container handling vehicles 201,301,401 in a second direction Y which is perpendicular to the first direction X. Containers 106 stored in the columns 105 are accessed by the container handling vehicles 201,301,401 through access openings 112 in the rail system 108. The container handling vehicles 201,301,401 can move laterally above the storage columns 105, i.e. in a plane which is parallel to the horizontal X-Y plane.

The upright members 102 of the framework structure 100 may be used to guide the storage containers during raising of the containers out from and lowering of the containers into the columns 105. The stacks 107 of containers 106 are typically self-supporting.

Each prior art container handling vehicle 201,301,401 comprises a vehicle body 201a, 301a, 401a and first and second sets of wheels 201b, 201c, 301b, 301c,401b,401c which enable the lateral movement of the container handling vehicles 201,301,401 in the X direction and in the Y direction, respectively. In FIGS. 2, 3 and 4 two wheels in each set are fully visible. The first set of wheels 201b, 301b,401b is arranged to engage with two adjacent rails of the first set 110 of rails, and the second set of wheels 201c, 301c,401c is arranged to engage with two adjacent rails of the second set 111 of rails. At least one of the sets of wheels 201b, 201c, 301b,301c,401b,401c can be lifted and lowered, so that the first set of wheels 201b, 301b,401b and/or the second set of wheels 201c, 301c,401c can be engaged with the respective set of rails 110, 111 at any one time.

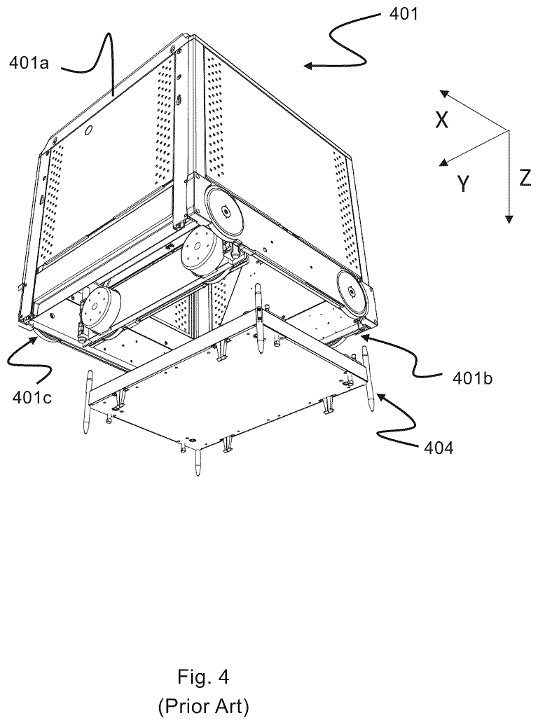

Each prior art container handling vehicle 201,301,401 also comprises a lifting device for vertical transportation of storage containers 106, e.g. raising a storage container 106 from, and lowering a storage container 106 into, a storage column 105. The lifting device comprises one or more gripping/engaging devices which are adapted to engage a storage container 106, and which gripping/engaging devices can be lowered from the vehicle 201,301,401 so that the position of the gripping/engaging devices with respect to the vehicle 201,301,401 can be adjusted in a third direction Z which is orthogonal the first direction X and the second direction Y. Parts of the gripping device of the container handling vehicles 301,401 are shown in FIGS. 3 and 4 indicated with reference number 304,404. The gripping device of the container handling device 201 is located within the vehicle body 201a in FIG. 2 and is thus not shown.

Conventionally, and also for the purpose of this application, Z=1 identifies the uppermost layer available for storage containers below the rails 110, 111, i.e. the layer immediately below the rail system 108, Z=2 the second layer below the rail system 108, Z=3 the third layer etc. In the exemplary prior art disclosed in FIG. 1, Z=7 identifies the lowermost, bottom layer of storage containers. Similarly, X=1 . . . n and Y=1 . . . n identifies the position of each storage column 105 in the horizontal plane. Consequently, as an example, and using the Cartesian coordinate system X, Y, Z indicated in FIG. 1, the storage container identified as 106′ in FIG. 1 can be said to occupy storage position X=17, Y=1, Z=5. The container handling vehicles 201,301,401 can be said to travel in layer Z=0, and each storage column 105 can be identified by its X and Y coordinates. Thus, the storage containers shown in FIG. 1 extending above the rail system 108 are also said to be arranged in layer Z=0.

The storage volume of the framework structure 100 has often been referred to as a grid 104, where the possible storage positions within this grid are referred to as storage cells. Each storage column may be identified by a position in an X- and Y-direction, while each storage cell may be identified by a container number in the X-, Y- and Z-direction.

Each prior art container handling vehicle 201,301,401 comprises a storage compartment or space for receiving and stowing a storage container 106 when transporting the storage container 106 across the rail system 108. The storage space may comprise a cavity arranged internally within the vehicle body 201a,401a as shown in FIGS. 2 and 4 and as described in e.g. WO2015/193278A1 and WO2019/206487A1, the contents of which are incorporated herein by reference.

FIG. 3 shows an alternative configuration of a container handling vehicle 301 with a cantilever construction. Such a vehicle is described in detail in e.g. NO317366, the contents of which are also incorporated herein by reference.

The cavity container handling vehicle 201 shown in FIG. 2 may have a footprint that covers an area with dimensions in the X and Y directions which is generally equal to the lateral extent of a storage column 105, e.g. as is described in WO2015/193278A1, the contents of which are incorporated herein by reference. The term ‘lateral’ used herein may mean ‘horizontal’.

Alternatively, the cavity container handling vehicles 401 may have a footprint which is larger than the lateral area defined by a storage column 105 as shown in FIGS. 1 and 4, e.g. as is disclosed in WO2014/090684A1 or WO2019/206487A1.

The rail system 108 typically comprises rails with grooves in which the wheels of the vehicles run. Alternatively, the rails may comprise upwardly protruding elements, where the wheels of the vehicles comprise flanges to prevent derailing. These grooves and upwardly protruding elements are collectively known as tracks. Each rail may comprise one track, or each rail 110,111 may comprise two parallel tracks. In other rail systems 108, each rail in one direction (e.g. an X direction) may comprise one track and each rail in the other, perpendicular direction (e.g. a Y direction) may comprise two tracks. Each rail 110,111 may also comprise two track members that are fastened together, each track member providing one of a pair of tracks provided by each rail.

WO2018/146304A1, the contents of which are incorporated herein by reference, illustrates a typical configuration of a rail system 108 comprising rails and parallel tracks in both X and Y directions.

In the framework structure 100, a majority of the columns 105 are storage columns 105, i.e. columns 105 where storage containers 106 are stored in stacks 107. However, some columns 105 may have other purposes. In FIG. 1, columns 119 and 120 are such special-purpose columns used by the container handling vehicles 201,301,401 to drop off and/or pick up storage containers 106 so that they can be transported to an access station (not shown) where the storage containers 106 can be accessed from outside of the framework structure 100 or transferred out of or into the framework structure 100. Within the art, such a location is normally referred to as a ‘port’ and the column in which the port is located may be referred to as a ‘port column’ 119,120. The transportation to the access station may be in any direction, that is horizontal, tilted and/or vertical. For example, the storage containers 106 may be placed in a random or dedicated column 105 within the framework structure 100, then picked up by any container handling vehicle and transported to a port column 119,120 for further transportation to an access station. The transportation from the port to the access station may require movement along various different directions, by means such as delivery vehicles, trolleys or other transportation lines. Note that the term ‘tilted’ means transportation of storage containers 106 having a general transportation orientation somewhere between horizontal and vertical.

In FIG. 1, the first port column 119 may for example be a dedicated drop-off port column where the container handling vehicles 201,301,401 can drop off storage containers 106 to be transported to an access or a transfer station, and the second port column 120 may be a dedicated pick-up port column where the container handling vehicles 201,301,401 can pick up storage containers 106 that have been transported from an access or a transfer station.

The access station may typically be a picking or a stocking station where product items are removed from or positioned into the storage containers 106. In a picking or a stocking station, the storage containers 106 are normally not removed from the automated storage and retrieval system 1, but are returned into the framework structure 100 again once accessed.

A port can also be used for transferring storage containers to another storage facility (e.g. to another framework structure or to another automated storage and retrieval system), to a transport vehicle (e.g. a train or a lorry), or to a production facility.

A conveyor system comprising conveyors is normally employed to transport the storage containers between the port columns 119,120 and the access station.

If the port columns 119, 120 and the access station are located at different levels, the conveyor system may comprise a lift device with a vertical component for transporting the storage containers 106 vertically between the port column 119,120 and the access station.

The conveyor system may be arranged to transfer storage containers 106 between different framework structures, e.g. as is described in WO2014/075937A1, the contents of which are incorporated herein by reference.

When a storage container 106 stored in one of the columns 105 disclosed in FIG. 1 is to be accessed, one of the container handling vehicles 201,301,401 is instructed to retrieve the target storage container 106 from its position and transport it to the drop-off port column 119. This operation involves moving the container handling vehicle 201,301,401 to a location above the storage column 105 in which the target storage container 106 is positioned, retrieving the storage container 106 from the storage column 105 using the container handling vehicle's 201,301,401 lifting device (not shown), and transporting the storage container 106 to the drop-off port column 119. If the target storage container 106 is located deep within a stack 107, i.e. with one or a plurality of other storage containers 106 positioned above the target storage container 106, the operation also involves temporarily moving the above-positioned storage containers prior to lifting the target storage container 106 from the storage column 105. This step, which is sometimes referred to as “digging” within the art, may be performed with the same container handling vehicle that is subsequently used for transporting the target storage container to the drop-off port column 119, or with one or a plurality of other cooperating container handling vehicles. Alternatively, or in addition, the automated storage and retrieval system 1 may have container handling vehicles 201,301,401 specifically dedicated to the task of temporarily removing storage containers 106 from a storage column 105. Once the target storage container 106 has been removed from the storage column 105, the temporarily removed storage containers 106 can be repositioned into the original storage column 105. However, the removed storage containers 106 may alternatively be relocated to other storage columns 105.

When a storage container 106 is to be stored in one of the columns 105, one of the container handling vehicles 201,301,401 is instructed to pick up the storage container 106 from the pick-up port column 120 and transport it to a location above the storage column 105 where it is to be stored. After any storage containers 106 positioned at or above the target position within the stack 107 have been removed, the container handling vehicle 201,301,401 positions the storage container 106 at the desired position. The removed storage containers 106 may then be lowered back into the storage column 105, or relocated to other storage columns 105.

For monitoring and controlling the automated storage and retrieval system 1, e.g. monitoring and controlling the location of respective storage containers 106 within the framework structure 100, the content of each storage container 106; and the movement of the container handling vehicles 201,301,401 so that a desired storage container 106 can be delivered to the desired location at the desired time without the container handling vehicles 201,301,401 colliding with each other, the automated storage and retrieval system 1 comprises a control system 500 which typically is computerized and which typically comprises a database for keeping track of the storage containers 106.

U.S. Pat. No. 9,403,196 describes a system for cleaning and decontamination of transport and storage boxes used for semiconductor substrates or photomasks, where reactive gases in the air is be removed to prevent defects in the substrates or photomasks during transport and storage. Here, a decontamination chamber with fluid-tight walls is required to be able to create a vacuum inside the chamber.

One object of the present invention is to provide a disinfection station and disinfection method for disinfecting storage containers in the above automated storage and retrieval system.

One object of the present invention is that the disinfection should not reduce efficiency of the automated storage and retrieval system considerably.

### SUMMARY OF THE INVENTION

The present invention relates to a disinfection station for disinfection of a storage container in an automated storage and retrieval system, wherein the disinfection station comprises:
a frame comprising a support, by which the storage container is supported in a disinfection position during disinfection, and an opening through which the storage container is transported between a storage position in the automated storage and retrieval system and the disinfection position;
a disinfection system for applying a disinfection fluid to the storage container in the disinfection position.

In one aspect, the disinfection system is a disinfection system for applying a disinfection fluid to the storage container in the disinfection position.

Alternatively, the disinfection system is a disinfection system for applying ultraviolet radiation to the storage container in the disinfection position.

The term “disinfection” is used herein to describe a process of neutralizing germs and/or viruses. “Disinfection” is different from “cleaning” and “washing”, in which case the intention is to remove dust/particles. Preferably, a disinfection fluid is applied to the surface of the storage container. The disinfection fluid is a fluid which neutralizes germs and/or viruses and which is evaporating from the surface of the storage container in a relatively short period of time. One such fluid may contain alcohol as the main active ingredient. Another such fluid may contain chlorine as the main active ingredient.

In one aspect, the storage container is held stationary in the disinfection position while the disinfection system is applying the disinfection fluid. Alternatively, the storage container passing through the disinfection position while the disinfection system is applying the disinfection fluid.

In one aspect, the disinfection fluid applicator comprises a disinfection fluid applicator, a disinfection fluid container and a pump controlled to pump the disinfection fluid from the container to the applicator.

In one aspect, the pump is controlled to pump the disinfection fluid from the container to the applicator based on a predetermined condition. One such condition may be that a container handling vehicle has moved a storage container to the disinfection position. In this case, the pump may be controlled by a signal received from a central control system, the container handling vehicle etc. Another such condition may be that a storage container is present in the disinfection position. In this case, the pump may be controlled by a signal from a detector detecting whether or not a storage container is present in the disinfection position.

In one aspect, the disinfection fluid applicator comprises at least one nozzle. The disinfection fluid applicator may be is directed to an interior surface of the storage container. The disinfection fluid applicator may be directed to an exterior surface of the storage container.

In one aspect, the disinfection station comprises a dryer for removing disinfection fluid from the storage container after disinfection.

In one aspect, the dryer comprises a heater. In one aspect, the dryer comprises a fan.

In one aspect, the disinfection station comprises a scanner for scanning the storage container before and/or after disinfection.

In one aspect, the disinfection system is configured to perform disinfection only if the scanner reveals that the storage container is empty.

In one aspect, the scanner is used to detect whether or not a storage container is present in the disinfection position.

In one aspect, the scanner comprises a camera. In one aspect, the scanner is configured to automatically detect whether the storage container is empty or not and/or whether a storage container is present in the disinfection position or not.

In one aspect, the disinfection system comprises a control system for controlling the pump and/or the dryer.

In one aspect the control system is controlling the pump and/or the dryer based on information received by the scanner. Alternatively, or in addition, the control system may control the pump and/or the dryer based on information received from a central control system.

In one aspect, the opening is a top opening, wherein the storage container is lowered through the top opening to the support before disinfection and wherein the storage container is lifted up through the top opening from the support after disinfection.

In one aspect, the support is a platform on which a base of the storage container is supported during disinfection.

In one aspect, the disinfection station comprises a splash cover for protecting the surroundings from disinfection fluid. The splash cover may be secured to the frame. Alternatively, the splash cover may be secured to the floor or other structures adjacent to the frame.

The present invention also relates to a access station for presentation of a storage container retrieved from an automated storage and retrieval system to a picker, wherein the access station comprises:
a frame comprising a support, a first opening through which the storage container is received to the support and a second opening through which the storage container is transported between the support and a presentation position in which the storage container is presented to the picker;
   characterized in that the access station is also a disinfection station defined with a disinfection position for the storage container when supported by the support;
   wherein the access station comprises a disinfection system for disinfecting the storage container in the disinfection position.

In one aspect, the first opening is a top opening, wherein the storage container is lowered through the top opening to the support and wherein the storage container is lifted up through the top opening from the support.

In one aspect, the second opening is a side opening, wherein the storage container is moved laterally through the side opening.

In one aspect, the above features of the disinfection station are also features of the access station.

The present invention also relates to an automated storage and retrieval system comprising a framework structure, wherein the framework structure comprises:
upright members;
a storage volume comprising columns provided between the members, wherein storage containers are stackable in stacks within at least some of the columns;
a rail system provided on top of the members;
   wherein the automated storage and retrieval system comprises container handling vehicles moving horizontally on the rail system;
   wherein the automated storage and retrieval system comprises a disinfection station as defined above or an access as defined above, wherein the frame is provided within the framework structure;
   wherein one of the columns is a transportation column aligned above the opening;
   wherein the support is provided within in the transportation column;
   wherein the storage container is transported between the storage position and the disinfection position via the transportation column and the opening.

In one aspect, the storage container is transported between the storage position and the disinfection position via the transportation column and the opening by means of one of the container handling vehicles.

In one aspect, the disinfection fluid container is located in a column adjacent to the transportation column.

In one aspect, storage containers are stackable above the disinfection fluid container.

In one aspect, the frame is provided within the framework structure as a support for at least some of the upright members of the framework structure.

Preferably, the frame is supporting four upright members of the framework structure.

The present invention also relates to a method for disinfection of a storage container in an automated storage and retrieval system, wherein the method comprises the following steps:
receiving the storage container at a disinfection position;
applying a disinfection fluid to the storage container in the disinfection position; and
moving the storage container away from the disinfection position.

In one aspect, the step of receiving the storage container comprises the step of:
receiving the storage container from a first one of a storage position in the automated storage and retrieval system or a picking position of an access station.

In one aspect, the step of moving the storage container away from the support comprises the step of:
moving the storage container to a second one of the storage position in the automated storage and retrieval system or the picking position of an access station.

In one aspect, the method further comprises the step of:
drying the storage container after the application of the disinfection fluid.

In one aspect, the method further comprises the step of:
scanning the storage container before the application of the disinfection fluid, and
applying disinfection fluid to the storage container only if a scanner reveals that the storage container is empty.

Disinfection of storage containers may be particularly relevant for automated storage and retrieval systems in which groceries are stored.

Disinfection of storage containers may be particularly relevant for automated storage and retrieval systems with pick-up terminals, the pick-up terminals being an interface where customers can pick up pre-ordered products themselves.

### DETAILED DESCRIPTION BRIEF DESCRIPTION OF THE DRAWINGS

Following drawings are appended to facilitate the understanding of the invention. The drawings show embodiments of the invention, which will now be described by way of example only, where:

FIG. 1 is a perspective view of a framework structure of a prior art automated storage and retrieval system.

FIG. 2 is a perspective view of a prior art container handling vehicle having an internally arranged cavity for carrying storage containers therein.

FIG. 3 is a perspective view of a prior art container handling vehicle having a cantilever for carrying storage containers underneath.

FIG. 4 is a perspective view, seen from below, of a prior art container handling vehicle having an internally arranged cavity for carrying storage containers therein.

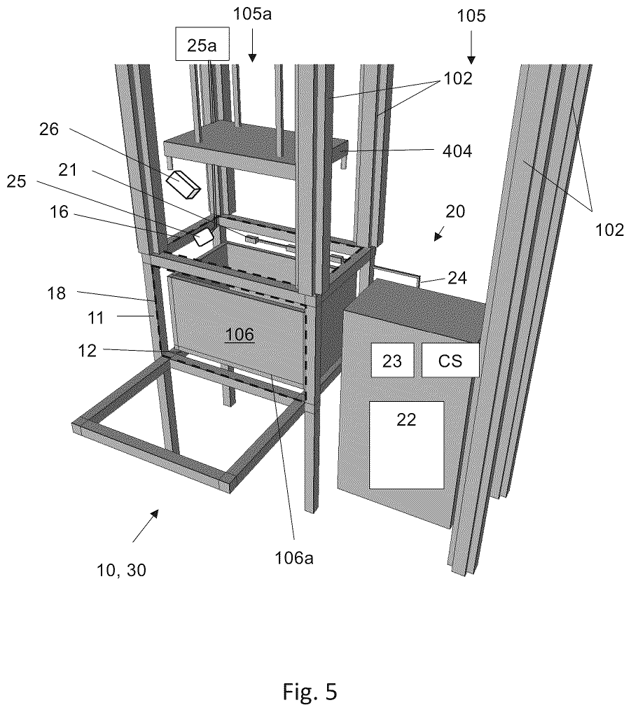

FIG. 5 is a perspective view of a disinfection station, which is integrated with an access station.

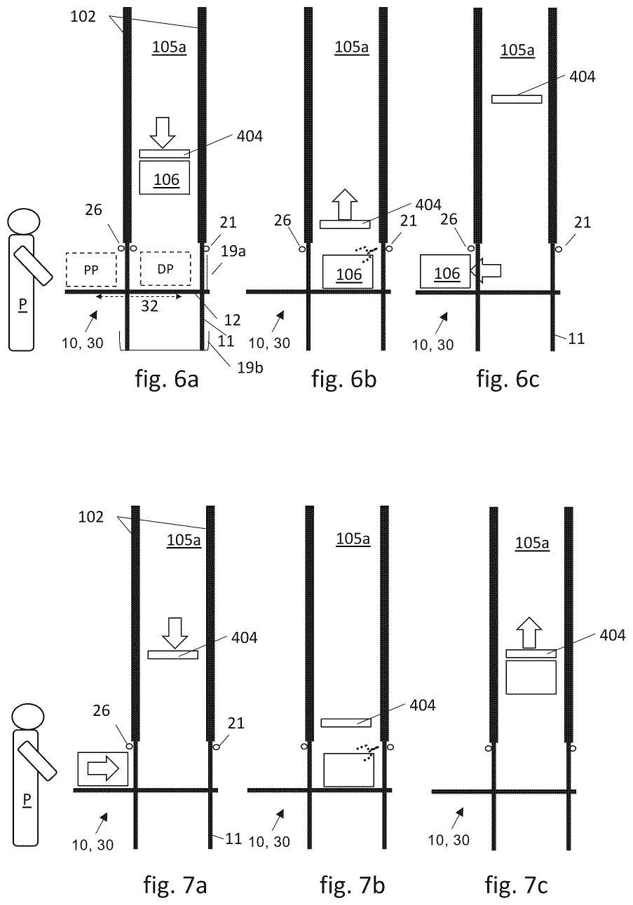

FIG. 6a-6c illustrate how a storage container is transported from a storage position, to a disinfection position and further to a picking position.

FIG. 7a-c illustrate how a storage container is transported from the picking position, to a disinfection position and further to a storage position.

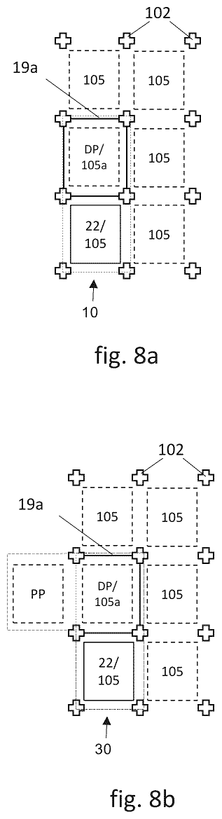

FIG. 8a illustrates a top view of the disinfection station schematically.

FIG. 8b illustrates a top view of the access station schematically.

### DETAILED DESCRIPTION OF THE INVENTION

In the following, embodiments of the invention will be discussed in more detail with reference to the appended drawings. It should be understood, however, that the drawings are not intended to limit the invention to the subject-matter depicted in the drawings.

The framework structure 100 of the automated storage and retrieval system 1 is constructed in a similar manner to the prior art framework structure 100 described above in connection with FIGS. 1-3. That is, the framework structure 100 comprises a number of upright members 102, and comprises a first, upper rail system 108 extending in the X direction and Y direction.

The framework structure 100 further comprises storage compartments in the form of storage columns 105 provided between the members 102 wherein storage containers 106 are stackable in stacks 107 within the storage columns 105.

The framework structure 100 can be of any size. In particular it is understood that the framework structure can be considerably wider and/or longer and/or deeper than disclosed in FIG. 1. For example, the framework structure 100 may have a horizontal extent of more than 700×700 columns and a storage depth of more than twelve containers.

In FIG. 1, a storage position for a storage container 106 is indicated as SP.

It is now referred to FIG. 5. Here, a disinfection station 10 is integrated with an access station 30.

The disinfection station 10 comprises a frame 11. The frame 11 is integrated with the framework structure 100, as vertical elements of the frame 11 is aligned with and is supporting members 102 of the framework structure 100. Hence, the area above frame 11 can be considered to be a column 105a through which a storage container 106 can be lowered and/or lifted by means of a gripping device 404 of a container handling vehicle 401. It should be noted that similar gripping devices of other container handling vehicles 201, 301 may also be used.

The frame comprises a support 12 forming a platform on which a base 106a of the storage container 106 can be supported. When a storage container 106 is supported by the support 12, the storage container 106 is considered to be in a disinfection position DP (shown in FIG. 6a), in which it can be disinfected.

The disinfection station 10 comprises a disinfection system 20 for applying a disinfection fluid to the storage container 106 in the disinfection position DP. The disinfection system 20 comprises a disinfection fluid applicator 21, a disinfection fluid container 22, a pump 23 and a fluid line 24, wherein the pump 23 is controlled to pump the disinfection fluid from the container 22 to the applicator 21 via fluid lines 24. The applicator comprises a number of nozzles from which disinfection fluid is sprayed on the exterior and interior surface of the storage container.

In the present embodiment, the disinfection fluid is a fluid containing ethanol as the main active disinfection ingredient. One advantage with ethanol is that it evaporates after relatively short time.

In the present embodiment, the pump 23 is controlled by a control system CS to apply a predetermined amount of disinfection fluid to the storage container.

The disinfection system 20 further comprises a dryer 25 for removing disinfection fluid from the storage container 106 after disinfection. The dryer 25 is also controlled by the control system CS. The dryer 25 may comprise a heater, a fan or a combination thereof. Such a dryer 25 will reduce the time for the evaporation of the ethanol from the surface of the storage container. The control system 25 is configured to control the dryer 25 to start after a predetermined time after the application of the disinfection fluid has ended.

The disinfection system 20 further comprises a scanner 26 for scanning the storage container 106 before and/or after disinfection. The scanner 26 is also controlled by the control system CS. In the present embodiment, the scanner 26 has two purposes. First, the purpose is to detect whether or not a storage container is present in the disinfection position DP. Second, the purpose is to detect whether or not the storage container in the disinfection position DP is empty or not. Hence, the control system CS may control the pump 23 to only spray disinfection fluid when an empty storage container is present in the disinfection position DP. In an alternative embodiment, a weight sensor may be used to detect if a storage container is present in the disinfection position DP and to determine whether or not the storage container is empty.

As indicated by a dashed rectangle in FIG. 5, the frame 11 has an top opening 16 through which a storage container 106 is transported from a storage position SP in one of the storage columns 105 in the automated storage and retrieval system 1 and to the disinfection position DP or from the disinfection position DP to the storage position SP in one of the storage columns 105 in the automated storage and retrieval system 1. As described above, this movement is typically performed by means of the gripping device 404 of one of the container handling vehicles.

As indicated by another dashed rectangle in FIG. 5, the frame 11 has a side opening 18 through which a storage container 106 can be moved to a presentation position PP (shown in FIG. 6a). In FIG. 6a, it is further shown a two-way dashed arrow denoted 32, which is referred to herein as an actuator 32 for moving the storage container between the disinfection position DP and the presentation position PP. Many such actuators exist in prior art access stations. The actuator 32 may comprise rollers rotated by means of a motor, wherein these rollers are also forming the base 12 on which the storage container 106 is supported during disinfection. Alternatively, the actuator 32 may comprise a linear actuator for pushing/pulling the storage container 106 between the disinfection position DP and the presentation position PP.

In FIG. 7a it is further shown that the disinfection station 10 comprises a splash cover 19a for protecting the surroundings from disinfection fluid. The splash cover 19a is here secured to the frame 11.

The disinfection station 10 may further comprise a collector 19 for collecting excessive fluid flowing down from the storage container 106, the splash cover 19a, the support 12 etc. As some storage containers 106 are provided with openings in their base, excessive fluid will flow from the inside of the storage container through these openings and down into the collector. The collector 19b may be provided in fluid communication with a drainage. The fluid may also be reused.

Dependent on the type of fluid used, the disinfection station 10 may comprise a ventilator 25a to remove evaporated disinfection fluid, to avoid or at least considerably reduce amount of fumes of disinfection fluid the picker P is exposed to. In the present embodiment, the ventilator 25a is a part of the dryer 25. Alternatively, the ventilator is separate dryer or even separate from the disinfection station 10.

Operation of Disinfection Station 10 Integrated with Access Station 30

The operation of the disinfection station 10 integrated with access station 30 will now be described with reference to FIG. 6a-6c. The access station 30 is further integrated in the framework structure 100 of the automated storage and retrieval system 1 of FIG. 1.

In FIG. 6a, the different positions in the access station 30 is indicated as dashed boxes indicated as the presentation position PP and the disinfection position DP. It is also shown that a gripping device 404 is lowering a storage container 106 through one column 105a of the framework structure 100 towards the disinfection position DP. It should be noted that the column 105a of the access station 30 is here a dedicated transportation column, and can therefore not be used for storage of storage containers.

In FIG. 6b, the storage container 106 is in the disinfection position DP and the gripping device 404 is moved upwardly again. The control system CS is detecting that an empty storage container 106 is present in the disinfection position DP by means of signals from the scanner 26 and the pump 23 is starting to pump disinfection fluid from the storage 22 to the applicator 25 via the fluid line 24. After a predetermined amount of fluid has been sprayed onto the surface of the storage container, the control system stops the pump. Then the control system CS is starting the dryer 25 to dry the storage container. Excessive fluid is collected by the collector 19b and excessive fumes may be removed by the dryer and/or ventilator.

In FIG. 6c, the storage container 106 is moved from the disinfection position DP to the presentation position PP by means of the actuator 32. The picker P may now supply products into the disinfected storage container before the storage container is moved back to the disinfection position DP, in which no disinfection is performed now, since the storage container 106 is no longer empty.

In FIG. 7a, the picker P has emptied the storage container 106 in the presentation position PP and the storage container 106 is moved from the presentation position PP to the disinfection position DP. As shown in FIG. 6a, a gripping device 404 is already preparing to pick up the storage container 106 from the disinfection position DP and is moving down the transportation column 105a.

In FIG. 7b, the control system CS is controlling the disinfection station 10 to perform disinfection and subsequent drying.

In FIG. 7c, the gripping device 404 is lifting the container 106 from the disinfection position DP and further to a storage position SP in the automated storage and retrieval system 1.

It is now referred to FIG. 8b, wherein the combined access station 30 and disinfection station 10 are shown from above. As in FIG. 5, FIG. 6a and FIG. 7a, the picking position PP is located outside of the framework structure 100 (i.e. not below or aligned with one of the columns 105, 105a). The footprint of the access station 30 is here two columns, the first one denoted as DP/105a and the second one denoted as 22/105. The first one, DP/105a is a transportation column. The second one, 22/105 is partially occupied with the fluid container 22 and possibly also the control system CS and the pump 23 and partially occupied with storage containers stacked above the fluid container 22.

It is now referred to FIG. 8a. Here, the disinfection station 10 is not integrated with an access station 30. Hence, there is no presentation position PP and no side opening 18. The storage container 106 is moved vertically down through the transport column 105a to the disinfection position DP and is then moved vertically up from the disinfection position DP. The footprint of the disinfection station 10 is here two columns, the first one denoted as DP/105a and the second one denoted as 22/105.

It is of course possible to locate the fluid container 22 and the pump 23 outside of the framework structure 100 and hence achieve a footprint of only one column (column DP/105a) of the framework structure.

It should further be noted that the central processing system 500 may register the date/time for each disinfection of the respective storage container, as each storage container has a unique ID stored in the system 500.

In the preceding description, various aspects of the delivery vehicle and the automated storage and retrieval system according to the invention have been described with reference to the illustrative embodiment. For purposes of explanation, specific numbers, systems and configurations were set forth in order to provide a thorough understanding of the system and its workings. However, this description is not intended to be construed in a limiting sense. Various modifications and variations of the illustrative embodiment, as well as other embodiments of the system, which are apparent to persons skilled in the art to which the disclosed subject matter pertains, are deemed to lie within the scope of the present invention.

1.-20. (canceled)

21. Disinfection station for disinfection of a storage container in an automated storage and retrieval system, wherein the disinfection station comprises:
a frame comprising:
a support, wherein the support is configured to support the storage container in a disinfection position during disinfection, and
an opening configured to allow the storage container to be transported between a storage position in the automated storage and retrieval system and the disinfection position;
a disinfection system for disinfecting the storage container in the disinfection position;
wherein the disinfection system comprises a disinfection fluid applicator, a disinfection fluid container, and a pump configured to pump the disinfection fluid from the container to the applicator;
wherein the opening is a top opening;
wherein the top opening is configured to allow the storage container to be:
lowered through the top opening to the support before disinfection; and
lifted up through the top opening from the support after disinfection.

22. Disinfection station according to claim 21, wherein the disinfection station comprises a splash cover for protecting the surroundings from disinfection fluid.

23. Disinfection station according to claim 21, wherein the disinfection station comprises a collector for collecting excessive disinfection fluid.

24. Disinfection station according to claim 21, wherein the disinfection station comprises a dryer for removing disinfection fluid from the storage container after disinfection.

25. Disinfection station according to claim 21, wherein the disinfection station comprises a scanner for scanning the storage container before and/or after disinfection.

26. Disinfection station according to claim 25, wherein the disinfection system is configured to perform disinfection only if the scanner reveals that the storage container is empty.

27. Disinfection station according to claim 21, wherein the disinfection system comprises a control system for controlling the pump and/or the dryer.

28. Disinfection station according to claim 21, wherein the frame comprises a second opening configured to allow the storage container to be transported between the support and a presentation position in which the storage container is presented to a picker of the automated storage and retrieval system, wherein the second opening is a side opening configured to allow the storage container to be moved laterally through the side opening.

29. Automated storage and retrieval system comprising a framework structure, wherein the framework structure comprises:
upright members;
a storage volume comprising columns provided between the members, wherein storage containers are stackable in stacks within at least some of the columns;
a rail system provided on top of the members;
wherein the automated storage and retrieval system further comprises:
container handling vehicles configured to move horizontally on the rail system; and
a disinfection station according to claim 1, wherein the frame is provided within the framework structure;
wherein one of the columns is a transportation column aligned above the opening, the support is provided within in the transportation column, and the transportation column is configured to allow the storage container to be transported between the storage position and the disinfection position via the transportation column and the opening.

30. Automated storage and retrieval system according to claim 29, wherein the container handling vehicles are configured to transport the storage container between the storage position and the disinfection position via the transportation column and the opening.

31. Automated storage and retrieval system according to claim 29, wherein the disinfection fluid container is located in a column adjacent to the transportation column.

32. Automated storage and retrieval system according to claim 29, wherein storage containers are stackable above the disinfection fluid container.
